(19) United States
(12) Patent Application Publication
(10) Pub. No.: US 2026/0167599 A1
(43) Pub. Date: Jun. 18, 2026

(54) PREPARATION METHOD AND USE OF LOW-POLARITY CHENOPODIUM QUINOA SAPONIN MONOMER

(71) Applicant: Shandong Second Medical University, Weifang City (CN)

(72) Inventors: Peng XUE, Weifang City (CN); Yan WANG, Weifang City (CN); Yi HE, Weifang City (CN)

(73) Assignee: Shandong Second Medical University, Weifang City (CN)

(21) Appl. No.: 19/309,770

(22) Filed: Aug. 26, 2025

(30) Foreign Application Priority Data
Dec. 17, 2024 (CN) 202411858670.7

(51) Int. Cl. C07C 67/56

(52) U.S. Cl. CPC C07C 67/56

(57) ABSTRACT

Provided are a preparation method and use of a low-polarity Chenopodium quinoa saponin monomer. The preparation method includes: S1, subjecting a Chenopodium quinoa bran to heating treatment, air-drying a resulting heat-treated bran, crushing a resulting air-dried Chenopodium quinoa bran and sieving a resulting crushed Chenopodium quinoa bran, subjecting a resulting sieved Chenopodium quinoa bran to extraction, then subjecting an obtained extract to a first concentration, subjecting the concentrate to saponin purification to obtain an eluate, subjecting the eluate to a second concentration, then spray-drying a concentrated solution to obtain a Chenopodium quinoa saponin mixture; and S2, subjecting the Chenopodium quinoa saponin mixture to gradient elution through a reverse-phase chromatographic column, collecting an eluate after elution with the 80% aqueous methanol solution, concentrating and drying the eluate, obtaining the low-polarity Chenopodium quinoa saponin monomer, namely a phytolaccagenic acid with a purity of not less than 90%.

### RELATED APPLICATIONS

This patent application claims the benefit and priority of Chinese Patent Application No. 202411858670.7 filed with the China National Intellectual Property Administration on Dec. 17, 2024, the disclosure of which is incorporated by reference herein in its entirety for all purposes.

### FIELD

The present disclosure belongs to the technical field of Chenopodium quinoa extraction, and in particular relates to a preparation method and use of a low-polarity Chenopodium quinoa saponin monomer.

### BACKGROUND

Chenopodium quinoa is a nutrient-rich grain native to South America, known as a “superfood”. Chenopodium quinoa is rich in bioactive components such as polyphenols, saponins, and flavonoids. Review studies have shown that Chenopodium quinoa saponins exhibit bacteriostatic activity. Chinese patent CN106176847A discloses a Chenopodium quinoa saponin having enhanced bacteriostatic effect and a preparation method thereof, where the Chenopodium quinoa saponin prepared by using alkali treatment heating can inhibit oral bacteria. However, the preparation process requires the use of sodium hydroxide, which is prone to pose risks to personnel, and the prepared saponin component is a mixture with unclear active substances.

Foodborne pathogenic bacteria are pathogenic bacteria that can cause food poisoning or use food as a transmission medium, and the foodborne pathogenic bacteria can directly or indirectly contaminate food and water sources. Oral infection in humans can lead to intestinal infectious diseases, food poisoning, and the prevalence of livestock and poultry infectious diseases. With the overuse of antibiotics, drug resistance of bacteria has been continuously increasing.

Therefore, there is a need to develop new drugs to inhibit the foodborne pathogenic bacteria.

### SUMMARY OF THE INVENTION

The present disclosure is intended to provide a preparation method and use of a low-polarity Chenopodium quinoa saponin monomer to solve the problem mentioned in the background described above.

In order to achieve the technical objects as described above, the present disclosure provides a method for preparing a low-polarity Chenopodium quinoa saponin monomer, comprising:

S1, weighing Chenopodium quinoa bran, subjecting the Chenopodium quinoa bran to heating treatment with superheated steam at a temperature of 110° C. to 120° C. for 1 h to 2 h, air-drying a resulting heat-treated bran to have a water content of not greater than 10%, crushing a resulting air-dried Chenopodium quinoa bran and sieving a resulting crushed Chenopodium quinoa bran by passing through a 40-mesh sieve, subjecting a resulting sieved Chenopodium quinoa bran to extraction with 70% ethanol at 60° C. for 0.5 h, then subjecting an obtained extract to a first concentration in a vacuum decompression concentration tank until a concentrate has no alcohol taste, subjecting the concentrate obtained from the first concentration to saponin purification by using HP-20 macroporous resin with a volume 15 times to 30 times that of the concentrate to obtain an eluate, subjecting the eluate to a second concentration again in the vacuum decompression concentration tank and recovering ethanol, then spray-drying a concentrated solution obtained from the second concentration at a temperature of 100° C. to 120° C. to obtain a Chenopodium quinoa saponin mixture; and

S2, subjecting the Chenopodium quinoa saponin mixture obtained in S1 to gradient elution with a 20% (volume percentage) aqueous methanol solution, a 40% (volume percentage) aqueous methanol solution, and a 80% (volume percentage) aqueous methanol solution sequentially through a reverse-phase chromatographic column, collecting an eluate after elution with 80% aqueous methanol solution, concentrating and drying the eluate by using a rotary evaporator and recovering methanol, obtaining the low-polarity Chenopodium quinoa saponin monomer, namely a phytolaccagenic acid with a purity of not less than 90%.

In some embodiments, in the S1, an amount of the 70% ethanol is 10 times to 15 times a mass of the resulting crushed Chenopodium quinoa bran; where the 70% ethanol refers to an aqueous ethanol solution with a volume percentage of 70%.

In some embodiments, in the S1, during the first concentration process, the vacuum decompression concentration tank is at a pressure of 0.09 MPa and a temperature of 40° C. to 50° C.; and during the second concentration, the vacuum decompression concentration tank is at a pressure of 0.09 MPa and a temperature of 40° C. to 50° C.

In some embodiments, in the S1, the saponin purification is conducted by: injecting the concentrate obtained from the first concentration into a chromatographic column filled with the HP-20 macroporous resin, standing and adsorbing for 8 h to 12 h; rinsing the chromatographic column with 2 BV of distilled water, and then rinsing the chromatographic column with 2 BV of 95% aqueous ethanol solution; and collecting the eluate.

In some embodiments, in the S2, the reverse-phase chromatographic column is filled with a reverse-phase silica gel packing, with a filling amount of the reverse-phase silica gel packing being 15 times to 30 times a mass of the Chenopodium quinoa saponin mixture.

In some embodiments, in the S2, the rotary evaporator is at a temperature of 40° C. to 60° C. and a pressure of 0.09 MPa.

The present disclosure also provides use of the low-polarity Chenopodium quinoa saponin monomer in preparation of drugs against foodborne pathogenic bacteria.

The present disclosure provides a method for preparing a low-polarity Chenopodium quinoa saponin monomer, where saponin components are enriched, separated and purified after direct heating treatment and air-drying of Chenopodium quinoa bran. Through high-heat conversion, the low-polarity Chenopodium quinoa saponin monomer QPhy is obtained in a purified extract, with a purity being 90%.

In vitro bacteriostatic experiments have verified that the low-polarity Chenopodium quinoa saponin monomer QPhy obtained by the present disclosure can inhibit the growth of foodborne pathogenic bacteria. In vivo bacteriostatic experiments have verified that the low-polarity Chenopodium quinoa saponin QPhy exhibits a protective effect on mice infected with lethal Listeria monocytogenes. The Chenopodium quinoa saponin monomer QPhy exhibits a good bacteriostatic effect on foodborne pathogenic bacteria and can be used as a drug against foodborne pathogenic bacteria, and can also be used in food industry as an additive, flavor regulator, and preservative to realize the high-value utilization of Chenopodium quinoa.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

To illustrate the technical solutions in the embodiments of the present disclosure or prior art more clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without inventive efforts.

FIG. 1A is a molecular diagram that presents the structural formula of QPhy;

FIG. 1B shows high-performance liquid chromatograms of the Chenopodium quinoa saponin obtained by conventional treatment;

FIG. 1C shows the low-polarity Chenopodium quinoa saponin monomer QPhy obtained by high-heat treatment in Example 1;

FIG. 2 shows a high-resolution mass spectrogram of the low-polarity Chenopodium quinoa saponin monomer QPhy in Example 1;

FIG. 3A shows the cell survival rate results of the low-polarity Chenopodium quinoa saponin monomer Qphy group and the Chenopodium quinoa saponin QPhy3 group, and FIG. 3B shows the cell count results of the 125 μg/mL low-polarity Chenopodium quinoa saponin monomer Qphy group, the 125 μg/mL Chenopodium quinoa saponin QPhy3 group, and the blank group;

FIG. 4A shows a diagram of results of bacteriostatic effects on Bacillus cereus in the in vitro bacteriostatic experiment in Example 5, and FIG. 4B shows a diagram of results of bacteriostatic effects on Staphylococcus aureus in the in vitro bacteriostatic experiment in Example 5; and

FIG. 5 shows a diagram of results of the survival rate of bacteria-infected mice in the in vivo bacteriostatic experiment in Example 6.

### DETAILED DESCRIPTION

Various exemplary embodiments of the present disclosure are described in detail below. This detailed description should not be construed as limiting the present disclosure, but rather as a more detailed description of certain aspects, features, and embodiments of the present disclosure.

It should be understood that the terms used in the present disclosure are only for describing specific embodiments and are not intended to limit the present disclosure. In addition, numerical ranges in the present disclosure should be understood as specifically disclosing the upper and lower limits of the ranges and each intermediate value therebetween. Each smaller range between any stated values or intermediate values within a stated range and any other stated values or intermediate values within that stated range is also included in the present disclosure. The upper and lower limits of such smaller ranges may be independently included in or excluded from a range.

Unless otherwise indicated, all technical and scientific terms used herein have the same meanings as commonly understood by one of ordinary skill in the art to which the present disclosure belongs. Although the present disclosure describes only preferred methods and materials, any methods and materials similar or equivalent to those described herein may also be used in the practice or testing of the present disclosure. All documents mentioned in the description are incorporated by reference to disclose and describe methods and/or materials associated with the documents. In case of conflict with any incorporated document, the content of the description shall prevail.

It would be apparent to those skilled in the art that various modifications and changes can be made to the specific embodiments of the present description without departing from the scope or spirit of the present disclosure. Other embodiments derived from the description of the present disclosure will be apparent to those skilled in the art. The description and examples of the present disclosure are merely exemplary.

As used herein, terms such as “comprise”, “include”, “have”, “contain” are open-ended terms, meaning including but not limited to.

### Example 1

This example provided a method for preparing a low-polarity Chenopodium quinoa saponin monomer QPhy. The method was performed as follows:

S1, Chenopodium quinoa bran was weighed, and subjected to heating treatment with superheated steam at 120° C. for 1 h. A resulting heat-treated bran was air-dried to have a water content of not greater than 10% (mass percentage). A resulting air-dried Chenopodium quinoa bran was crushed. A resulting crushed Chenopodium quinoa bran was sieved by passing through a 40-mesh sieve. A resulting sieved Chenopodium quinoa bran was subjected to extraction with 70% aqueous ethanol solution at 60° C. for 0.5 h (where an amount of the 70% aqueous ethanol solution was 15 times a mass of the resulting crushed Chenopodium quinoa bran). An obtained extract was subjected to a first concentration in a vacuum decompression concentration tank at 0.09 MPa and 40° C. until a concentrate had no alcohol taste. The concentrate obtained from the first concentration was subjected to saponin purification by using HP-20 macroporous resin with a volume 15 times that of the concentrate to obtain an eluate. The eluate was subjected to a second concentration again in the vacuum decompression concentration tank and ethanol was recovered. The second concentration was conducted at a pressure of 0.09 MPa and a temperature of 50° C. A concentrated solution obtained from the second concentration was spray-dried at 100° C. to obtain a Chenopodium quinoa saponin mixture.

Specifically, saponin purification was conducted by injecting the concentrate obtained from the first concentration into a chromatographic column filled with the HP-20 macroporous resin, standing and absorbing for 8 h; rinsing the chromatographic column with 2 BV of distilled water, and then rinsing the chromatographic column with 2 BV of 95% (volume percentage) aqueous ethanol solution; and collecting the eluate.

S2, the Chenopodium quinoa saponin mixture obtained in S1 was subjected to gradient elution with 20%, 40%, and 80% aqueous methanol solutions sequentially through a reverse-phase chromatographic column. An eluate after elution with 80% aqueous methanol solution was collected. The eluate was subjected to concentration and drying by using a rotary evaporator and methanol was recovered, obtaining the low-polarity Chenopodium quinoa saponin monomer, namely a low-polarity phytolaccagenic acid with a purity of not less than 90%, designated Chenopodium quinoa saponin monomer QPhy. In the S2, the reverse-phase chromatographic column was filled with a reverse-phase silica gel packing, with a filling amount of the reverse-phase silica gel packing being 15 times a mass of the Chenopodium quinoa saponin mixture, and the rotary evaporator was at a temperature of 40° C. and a pressure of 0.09 MPa.

In this example, high-performance liquid chromatography analysis was conducted on the low-polarity Chenopodium quinoa saponin monomer QPhy prepared as above and a Chenopodium quinoa saponin obtained by conventional treatment, respectively.

A treatment method for the Chenopodium quinoa saponin obtained by the conventional treatment was conducted as follows: 1 μg of Chenopodium quinoa bran was weighed, and then 10 mL of 70% (volume percentage) aqueous methanol solution was added thereto. A resulting mixture was subjected to ultrasonic extraction at an ultrasonic power of 700 w for 30 min. A resulting supernatant was taken and passed through a 0.45-μm filter membrane, and then subjected to the high-performance liquid chromatography analysis.

A treatment method for the QPhy monomer was conducted as follows: 2 mg of the low-polarity Chenopodium quinoa saponin monomer QPhy prepared in this example was accurately weighed. 1 mL of 70% aqueous methanol solution was added thereto for full dissolving the QPhy. A resulting mixture was passed through a 0.45-μm filter membrane, and then subjected to the high-performance liquid chromatography analysis.

The high-performance liquid chromatography were as follows:
chromatographic column: YMC ODS-Pack, 4.6 mm×250 mm, film thickness: 5 μm;
column oven temperature: 25° C.; detection wavelength: 202 nm; flow rate: 1 mL/min.
mobile phase A: ultrapure water; mobile phase B: acetonitrile;
Elution conditions were as follows:
0 to 5 min, with a volume percentage of the mobile phase B ranging from 0 to 10%;
5 min to 10 min, with a volume percentage of the mobile phase B ranging from 10% to 20%;
10 min to 15 min, with a volume percentage of the mobile phase B ranging from 20% to 28%;
15 min to 35 min, with a volume percentage of the mobile phase B ranging from 28% to 40%;
35 min to 40 min, with a volume percentage of the mobile phase B ranging from 40% to 60%; 40 min to 50 min, with a volume percentage of the mobile phase B ranging from 60% to 70%;
50 min to 60 min, with a volume percentage of the mobile phase B remaining 70%; and 60 min to 65 min, with a volume percentage of the mobile phase B ranging from 70% to 10%.

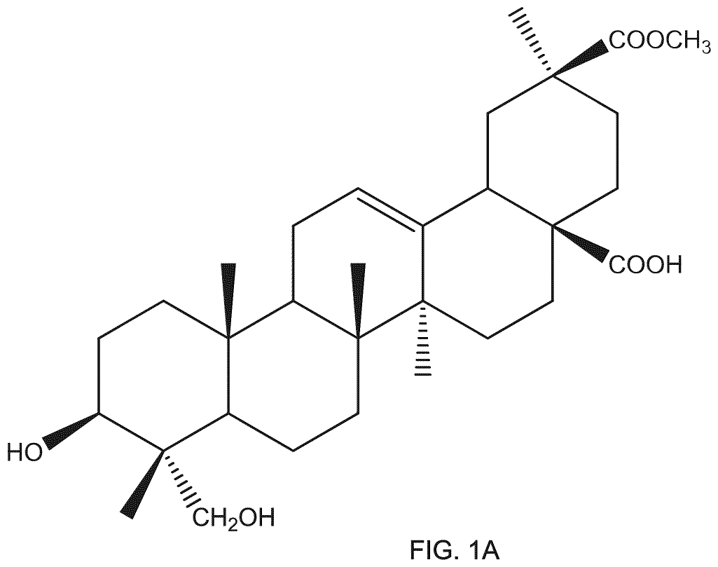

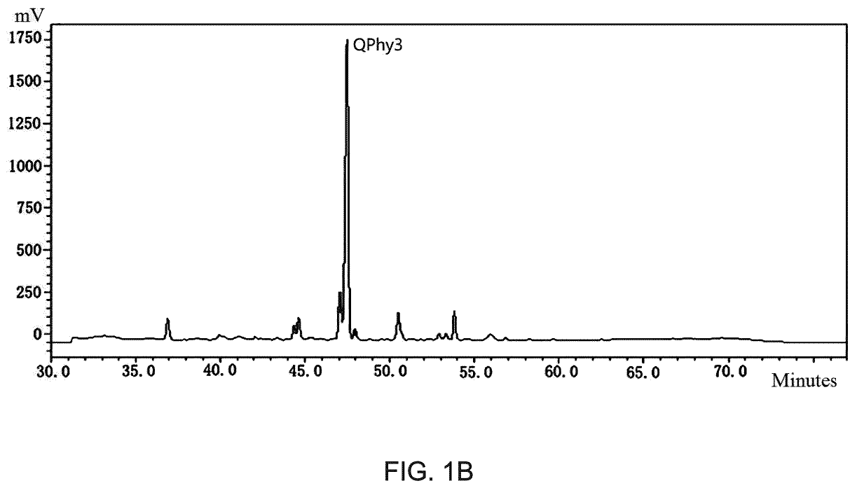

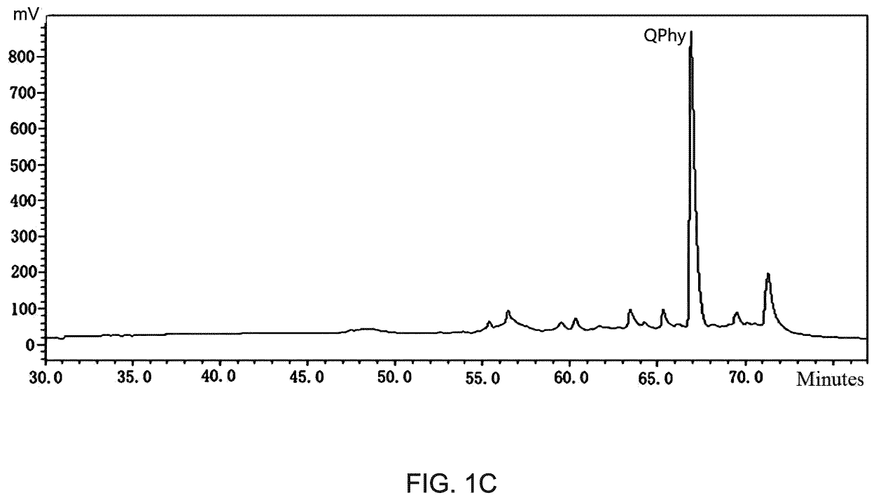

FIGS. 1B to 1C show high-performance liquid chromatograms of the Chenopodium quinoa saponin obtained by conventional treatment and the low-polarity Chenopodium quinoa saponin monomer QPhy prepared in this example, where FIG. 1B shows the high-performance liquid chromatogram of the Chenopodium quinoa saponin from the conventional treatment, and FIG. 1C shows the high-performance liquid chromatogram of QPhy prepared in this example.

It can be seen from FIGS. 1B to 1C that the Chenopodium quinoa saponin from the conventional treatment is mainly polar monomer QPhy3; after pyrolysis, the low-polarity Chenopodium quinoa saponin monomer prepared in this example is mainly QPhy.

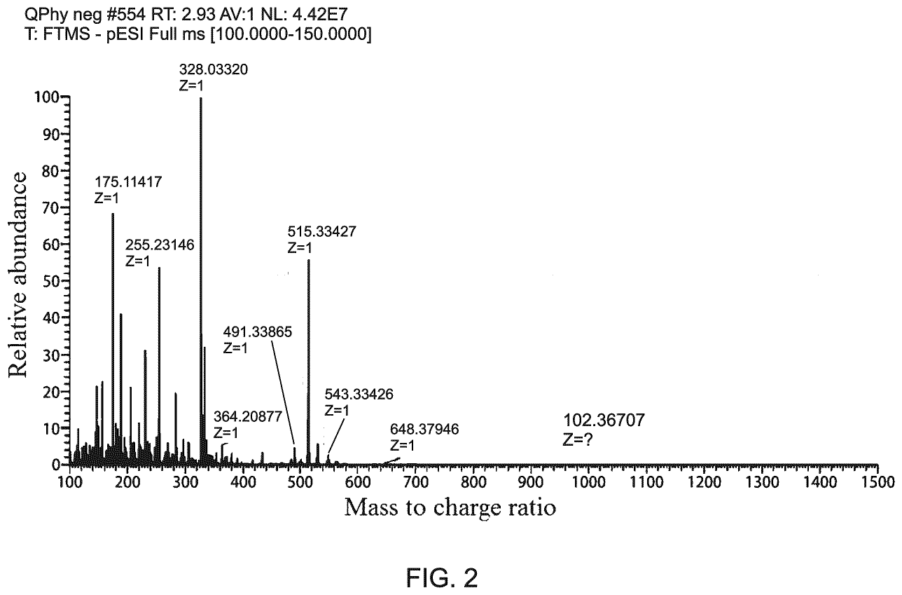

1 mg of Chenopodium quinoa saponin monomer QPhy was accurately weighed, a resulting weighed QPhy was fully dissolved in 5 mL of mass spectrometry-grade methanol to obtain a QPhy methanol solution with a concentration of 200 μg/mL. The Qphy methanol solution with the concentration of 200 μg/mL was directly determined by high-resolution mass spectrometry (Q Exactive mass spectrometer, Thermo), and a high-resolution mass spectrogram was obtained, as shown in FIG. 2.

Mass spectrometry conditions were as follows:
capillary voltage: −1500 V in negative ion mode;
skimmer voltage: 30 V;
ion source temperature: 320° C.;
desolvation gas temperature: 500° C.; scanning range: 80-1200 m/z.

As shown in FIG. 2, the molecular parent ion peak of the compound is 515.3347[M−H−].

The Chenopodium quinoa saponin monomer QPhy obtained in this example exhibits the following physicochemical properties: a white powder, showing a purplish-red color in vanillin-perchloric acid solution; insoluble in water, soluble in alcohols, belonging to saponin substances. The main 13CNMR chemical shift values (δ) of the Chenopodium quinoa saponin monomer QPhy are: C-28 (δ=179.80), C-30 (δ=177.36), C-13 (δ=147.37), C-12 (δ=122.74), C-3 (δ=72.46), C-23 (δ=65.93, no glycosidic bond). The compound is identified as QPhy, with the specific structural formula as shown in FIG. 1A.

### Example 2

This example provided a method for preparing a low-polarity Chenopodium quinoa saponin monomer QPhy. The method was performed as follows:

S1, Chenopodium quinoa bran was weighed, and subjected to heating treatment with superheated steam at 110° C. for 2 h. A resulting heat-treated bran was air-dried to have a water content of not greater than 10%. A resulting air-dried Chenopodium quinoa bran was crushed. A resulting crushed Chenopodium quinoa bran was sieved by passing through a 40-mesh sieve. A resulting sieved Chenopodium quinoa bran was subjected to extraction with 70% aqueous ethanol solution at 60° C. for 0.5 h (where an amount of the 70% aqueous ethanol solution was 10 times a mass of the resulting crushed Chenopodium quinoa bran). An obtained extract was subjected to a first concentration in a vacuum decompression concentration tank at 0.09 MPa and 50° C. until a concentrate had no alcohol taste. The concentrate obtained from the first concentration was subjected to saponin purification by using HP-20 macroporous resin with a volume 30 times that of the concentrate to obtain an eluate. The eluate was subjected to a second concentration again in the vacuum decompression concentration tank and ethanol was recovered. The second concentration was conducted at a pressure of 0.09 MPa and a temperature of 40° C. A concentrated solution obtained from the second concentration was spray-dried at 120° C. to obtain a Chenopodium quinoa saponin mixture.

Specifically, saponin purification was conducted by injecting the concentrate obtained from the first concentration into a chromatographic column filled with the HP-20 macroporous resin, standing and absorbing for 12 h; rinsing the chromatographic column with 2 BV of distilled water, and then rinsing the chromatographic column with 2 BV of 95% aqueous ethanol solution; and collecting the eluate.

S2, the Chenopodium quinoa saponin mixture obtained in S1 was subjected to gradient elution with 20%, 40%, and 80% aqueous methanol solutions sequentially through a reverse-phase chromatographic column. An eluate after elution with 80% aqueous methanol solution was collected. The eluate was subjected to concentration and drying by using a rotary evaporator and methanol was recovered, obtaining the low-polarity Chenopodium quinoa saponin monomer, namely a phytolaccagenic acid with a purity of not less than 90%, designated Chenopodium quinoa saponin monomer QPhy. In the S2, the reverse-phase chromatographic column was filled with a reverse-phase silica gel packing, with a filling amount of the reverse-phase silica gel packing being 30 times a mass of the Chenopodium quinoa saponin mixture, and the rotary evaporator was at a temperature of 60° C. and a pressure of 0.09 MPa.

### Example 3

In this example, Bacillus cereus was used as a test strain to determine the MIC and MBC values of the Chenopodium quinoa saponin monomer QPhy. A specific method was performed as follows:

Three test components of Chenopodium quinoa saponin QPhy3, Chenopodium quinoa saponin monomer QPhy, and positive control cefradine (purchased from Shandong Lukang Pharmaceutical Co., Ltd., China) were dissolved in DMSO respectively to prepare stock solutions at 100 mg/mL.

By using a two-fold dilution method, each stock solution was diluted with normal saline into drug solutions with decreasing concentration gradients: specifically, 100 mg/mL, 50 mg/mL, 25 mg/mL, 12.5 mg/mL, 6.25 mg/mL, 3.125 mg/mL, and 1.5625 mg/mL.

In a 96-well plate, 2 μL of each of three test component drug solutions with decreasing concentration gradients was added to each well, followed by 198 μL of bacterial suspension containing Bacillus cereus at 10⁴ CFU/mL, resulting in final concentrations of three test components in the 96-well plate of 1000 μg/mL, 500 μg/mL, 250 μg/mL, 125 μg/mL, 62.5 μg/mL, 31.25 μg/mL, and 15.625 μg/mL. For negative control wells, 2 μL of DMSO and 198 μL of bacterial suspension were added.

A prepared 96-well plate was incubated in an incubator for 12 h and observed. The test was considered invalid if there was no bacterial growth in the negative control wells. The minimum inhibitory concentration (MIC) of the saponin monomer against the test strain was determined as the lowest drug concentration at which no visible bacterial growth was observed by the naked eye, and the data were recorded. By using an inoculating loop, a drug with concentration at which no bacterial growth was observed was dipped and streaked onto agar medium. The lowest drug concentration at which no colonies grew was regarded as the minimum bactericidal concentration (MBC) of the saponin monomer against the test strain. The experimental results are shown in Table 1.

TABLE 1
MIC and MBC values of each test component
against Bacillus cereus

| Test sample | MIC (μg/mL) | MBC (μg/mL) |
|---|---|---|
| QPhy | 125.000 | 1000.000 |
| QPhy3 | —(Not detected) | —(Not detected) |
| Cefradine | 15.625 | 31.250 |

As shown in Table 1, among Chenopodium quinoa saponins, the Chenopodium quinoa saponin monomer QPhy exhibits an MIC value of 125.000 g/mL and an MBC value of 1000.000 μg/mL, while the Chenopodium quinoa saponin QPhy3 monomer shows no obvious bacteriostatic activity.

### Example 4

In this example, evaluation on the safety of the Chenopodium quinoa saponin monomer QPhy obtained in Example 1 was conducted, with a specific method being performed as follows:

1 mg of the Chenopodium quinoa saponin monomer QPhy obtained in Example 1 was accurately weighed and dissolved in 10 μL of DMSO. Then a resulting mixture was added into 990 μL of complete culture medium to prepare a 1 mg/mL saponin stock solution. Based on the MIC of the Chenopodium quinoa saponin monomer QPhy obtained in Example 3, the saponin stock solution was diluted with the complete culture medium to a concentration of 125 μg/mL for the Chenopodium quinoa saponin monomer QPhy.

GES-1 cells that had grown to 80% confluency in a cell flask were seeded into a 96-well plate at a concentration of 5×10′ cells/mL, and cultured in a cell incubator at 37° C. with 5% CO₂ for 24 hours. An original medium was discarded, cells were washed once with PBS, and then medium containing the Chenopodium quinoa saponin monomer QPhy at a concentration of 125 μg/mL was added. After culturing in a cell incubator at 37° C. with 5% CO₂ for 22 hours, complete culture medium containing CCK-8 reagent was added, and culture was continued for another 2 hours in the incubator at 37° C. with 5% CO₂, serving as drug-treated group.

Separately, GES-1 cells that had grown to 80% confluency in a cell flask were seeded into a 96-well plate at a concentration of 5×10¹ cells/mL, and cultured in a cell incubator at 37° C. with 5% CO₂ for 24 hours. An original medium was discarded, cells were washed once with PBS. After culturing in a cell incubator at 37° C. with 5% CO₂ for another 22 hours, complete culture medium containing CCK-8 reagent was added, and culture was continued for another 2 hours in the incubator at 37° C. with 5% CO₂, serving as non-drug-treated group.

Separately, a 96-well plate without GES-1 cells seeded was prepared, and cultured in a cell incubator at 37° C. with 5% CO₂ for 24 hours. An original medium was discarded, the 96-well plate was washed once with PBS. After culturing in a cell incubator at 37° C. with 5% CO₂ for another 22 hours, complete culture medium containing CCK-8 reagent was added, and culture was continued for another 2 hours in the incubator at 37° C. with 5% CO₂, serving as blank group.

For the above three groups of samples, 4 parallel wells were set for each group, and their OD₄₅₀ values were detected by using a microplate reader separately.

$$Calculations were performed by using the formula : Cell viability = [ OD ( drug - treated group ) - OD ( blank group ) ] / [ OD ( non - drug - treated group ) - OD ( blank group ) ] \times 100 \% .$$

Chenopodium quinoa saponin monomer Qphy at a concentration of 125 g/mL was diluted to Chenopodium quinoa saponin monomer Qphy at a concentration of 62.5 μg/mL.

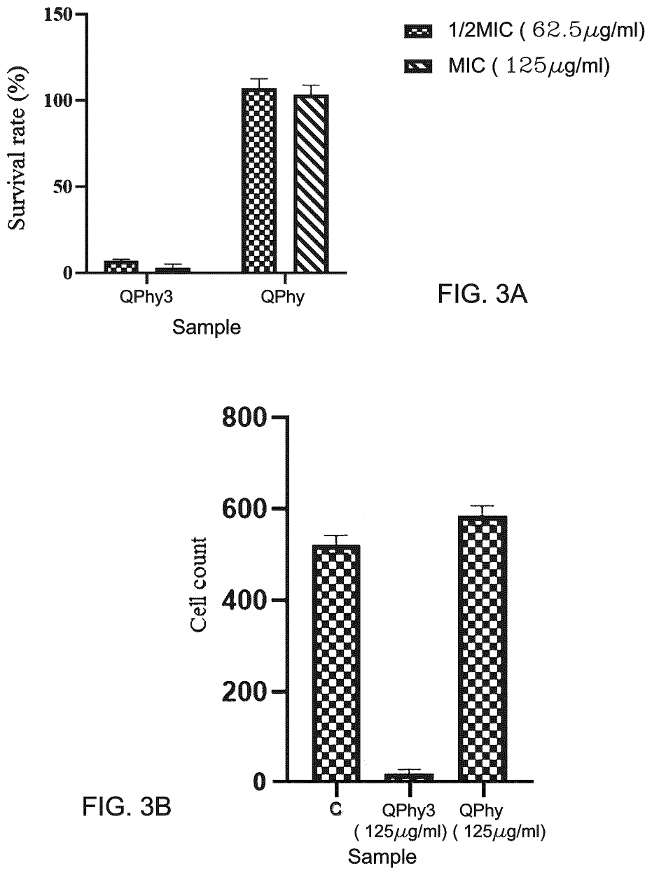

Five groups of GES-1 cells were prepared. Two groups of GES-1 cells were treated with Chenopodium quinoa saponin monomer Qphy at concentrations of 125 μg/mL and 62.5 μg/mL for 24 h, respectively. Another two groups of GES-1 cells were treated with Chenopodium quinoa saponin QPhy3 at concentrations of 125 μg/mL and 62.5 μg/mL for 24 h, respectively. The last group served as control group with no drug added. The survival rate of GES-1 cells in each group was detected by using a CCK-8 method. The results are shown in FIGS. 3A to 3B, where FIG. 3A shows the cell survival rate results of the Chenopodium quinoa saponin monomer Qphy group and the Chenopodium quinoa saponin QPhy3 group, and FIG. 3B shows the cell count results of 125 μg/mL Chenopodium quinoa saponin monomer Qphy group, 125 μg/mL Chenopodium quinoa saponin QPhy3 group, and the control group.

As can be seen from FIGS. 3A to 3B, compared with the control group, the Chenopodium quinoa saponin monomer QPhy slightly promotes the proliferation of GES-1 cells without toxicity. After treating the GES-1 cells with Chenopodium quinoa saponin QPhy3 at the corresponding concentrations for 24 h, the percentage of dead cells is much higher than that in the Chenopodium quinoa saponin monomer QPhy group.

### Example 5

This example verified that the low-polarity Chenopodium quinoa saponin monomer QPhy of the present disclosure inhibits the growth of foodborne pathogenic bacteria through in vitro bacteriostatic experiments. A specific method was performed as follows:

Staphylococcus aureus (purchased from Guangdong Microbial Culture Collection Center, China) and Bacillus cereus (purchased from Guangdong Microbial Culture Collection Center, China) were used as test strains, and a paper disk method was employed to study the bacteriostatic effect of saponins.

0.1 mL of bacterial suspension containing 1.5×10⁸ CFU/mL of Staphylococcus aureus and 0.1 mL of bacterial suspension containing 1.5×10⁸ CFU/mL of Bacillus cereus were each evenly spread over the entire surface of nutrient agar solid medium plates.

Chenopodium quinoa bran was weighed and crushed, and a resulting crushed Chenopodium quinoa bran was sieved by passing through a 40-mesh sieve. A resulting sieved Chenopodium quinoa bran was subjected to extraction with 70% aqueous ethanol solution at 60° C. for 0.5 h (where an amount of the 70% aqueous ethanol solution was 15 times a mass of the resulting crushed Chenopodium quinoa bran). An obtained extract was then subjected to a first concentration in a vacuum decompression concentration tank at 0.09 MPa and 40° C. until a concentrate had no alcohol taste. The concentrate was subjected to saponin purification by using HP-20 macroporous resin with a volume 15 times that of the concentrate to obtain an eluate. The eluate was subjected to a second concentration again in the vacuum decompression concentration tank and ethanol was recovered. The second concentration was conducted at a pressure of 0.09 MPa and a temperature of 50° C.). Then a concentrated solution obtained from the second concentration was spray-dried at 100° C. to obtain a pre-treatment crude Chenopodium quinoa saponin.

Specifically, the saponin purification was conducted by injecting the concentrate obtained from the first concentration into a chromatographic column filled with the HP-20 macroporous resin, standing and absorbing for 8 h; rinsing the HP-20 macroporous resin with 2 BV of distilled water, and then rinsing the chromatographic column with 2 BV of 95% aqueous ethanol solution; and collecting the eluate.

The pre-treatment crude Chenopodium quinoa saponin as obtained above was subjected to gradient elution with 20%, 40%, and 80% aqueous methanol solutions sequentially through a reverse-phase chromatographic column. An eluate after elution with 80% aqueous methanol solution was subjected to collecting, concentration and drying in sequence by using a rotary evaporator and methanol was recovered. After the concentration and drying, Chenopodium quinoa saponin QPhy3 was obtained. The reverse-phase chromatographic column was filled with a reverse-phase silica gel packing, with a filling amount of the reverse-phase silica gel packing being 15 times a mass of the pre-treatment crude Chenopodium quinoa saponin. The rotary evaporator was operated at a temperature of 40° C. and a pressure of 0.09 MPa.

The Chenopodium quinoa saponin mixture obtained in Example 1 was used as a post-treatment crude Chenopodium quinoa saponin. The pre-treatment crude Chenopodium quinoa saponin, the post-treatment crude Chenopodium quinoa saponin, Chenopodium quinoa saponin QPhy3, Chenopodium quinoa saponin monomer QPhy obtained in Example 1, penicillin (purchased from Shandong Lukang Pharmaceutical Co., Ltd., China), and cefradine were dissolved in 75% ethanol solutions separately to prepare sample solutions with a concentration of 5 mg/mL.

Seven paper disks, numbered 1 to 7, were prepared. Disk 1 was immersed in 10 μL of ethanol solution as negative control group, then taken out and air-dried. Disks 2 to 5 were respectively immersed in 10 μL of the sample solutions of the pre-treatment crude Chenopodium quinoa saponin, the post-treatment crude Chenopodium quinoa saponin, the Chenopodium quinoa saponin monomer QPhy3, and the Chenopodium quinoa saponin monomer QPhy, taken out, air-dried, and operation was repeated twice. Disks 6 to 7 were immersed in 10 L of the sample solutions of penicillin and cefradine respectively, then taken out and air-dried. After all disks were air-dried, they were placed on solid medium plates containing Staphylococcus aureus and incubated in a 37° C. constant temperature incubator. After 6 hours of incubation, the solid medium plates were taken out and photographed for observation.

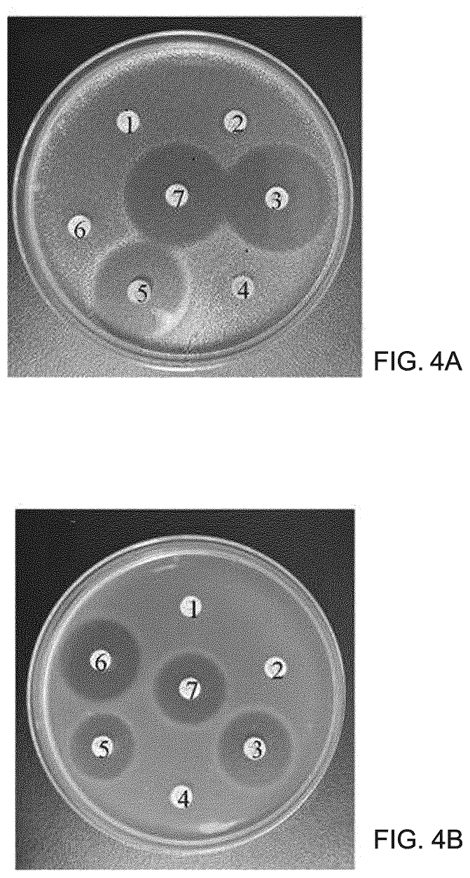

A test method using Bacillus cereus as test strains was the same as that for Staphylococcus aureus. Three parallel groups were set for each strain. The experimental results are shown in FIGS. 4A to 4B, where FIG. 4A shows the solid medium with Bacillus cereus, and FIG. 4B shows the solid medium with Staphylococcus aureus.

It can be seen from FIGS. 4A to 4B that crude Chenopodium quinoa saponin solution before treatment has no obvious inhibitory effect on Bacillus cereus and Staphylococcus aureus; crude Chenopodium quinoa saponin solution after treatment and the Chenopodium quinoa saponin monomer QPhy both show a certain inhibitory effect, while main Chenopodium quinoa saponin monomer QPhy3 in the crude Chenopodium quinoa saponin solution before treatment also has no obvious inhibitory effect on Bacillus cereus and Staphylococcus aureus.

Then, the diameters of the bacteriostatic zones of disks 1 to 7 were measured respectively, and the results are shown in Table 2.

TABLE 2
Diameter results of bacteriostatic zones

| | Diameter of bacteriostatic zone (mm) | |
|---|---|---|
| Test sample | Staphylococcus aureus | Bacillus cereus |
| Pre-treatment crude | Not detected | Not detected |
| Chenopodium quinoa | | |
| saponin | | |
| Post-treatment crude | 25.18 ± 0.51 | 19.37 ± 0.46 |
| Chenopodium quinoa | | |
| saponin | | |
| QPhy | 23.44 ± 0.66 | 17.75 ± 0.34 |
| QPhy3 | Not detected | Not detected |
| Penicillin | 25.22 ± 0.65 | Not detected |
| Cefradine | 23.28 ± 0.47 | 18.35 ± 0.38 |

### Example 6

This example verified the protective effect of the low-polarity Chenopodium quinoa saponin QPhy on mice infected with lethal Listeria monocytogenes (purchased from Guangdong Microbial Culture Collection Center, China) through an in vivo bacteriostatic experiment. A specific method was conducted as follows:

Thirty 6- to 8-week-old female C57BL/6J mice with stable weight gain, shiny fur after adaptive feeding, and weighing 18 g to 20 g were selected and grouped according to randomization principle into 5 groups with 6 mice in each group, namely blank group (Cont group), negative group (Neg group), penicillin-treated group (Pos group), pre-treatment crude Chenopodium quinoa saponin-treated group (QS group), and group treated by post-treatment crude Chenopodium quinoa saponin rich in Chenopodium quinoa saponin QPhy (QPhy group).

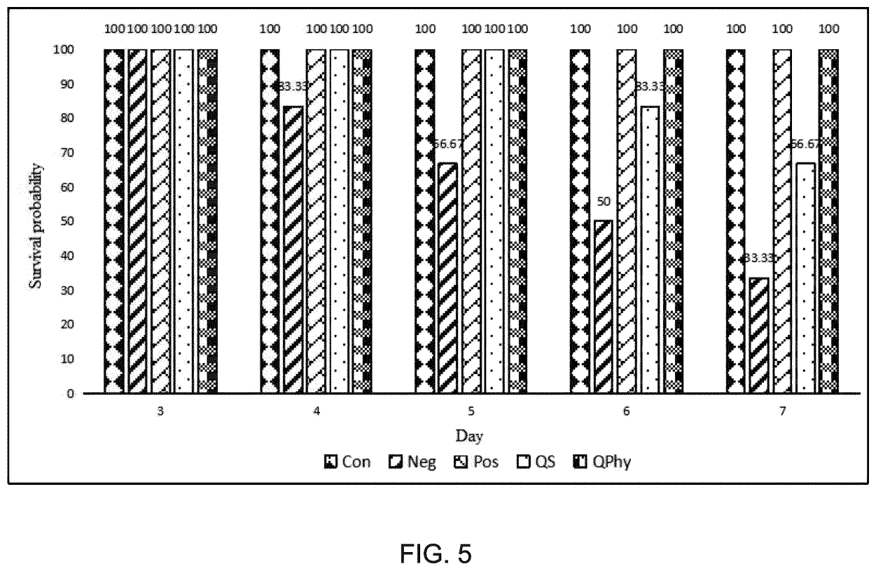

The QS group and the QPhy group were pre-treated by intragastric administration of aqueous solutions of pre-treatment crude Chenopodium quinoa saponin and post-treatment crude Chenopodium quinoa saponin at 5 mg/mL, respectively, for 14 days using the same volume, with an intragastric administration amount of 10 mL/kg/d. The other three groups received no pre-treatment. After 14 days, except for the blank group, the other groups were intraperitoneally injected with 0.1 mL of Listeria monocytogenes bacterial suspension at 106 CFU/mL. After infection, mice in QS and QPhy groups continued to receive intragastric treatment at the same dose as the pre-treatment. Mice in the positive group were intraperitoneally injected with 0.1 mL of penicillin injection at 50,000 units/0.1 mL, and negative and blank groups received no treatment, where all groups of mice were provided with sufficient water and feed, and their survival rates were observed. The experimental results are shown in FIG. 5.

As shown in FIG. 5, the survival rate of mice in the negative group is 33.3% within seven days, while the survival rates of blank and positive groups are 100% within seven days. The survival rate of the QS group is 66.6% within seven days, and that of the QPhy group is 100% within seven days. Therefore, the low-polarity Chenopodium quinoa saponin monomer QPhy has a significant preventive and protective effect on mice infected with Listeria monocytogenes.

In the present disclosure, the low-polarity Chenopodium quinoa saponin substance QPhy is obtained through high-heat steam transformation. The Chenopodium quinoa saponin monomer QPhy obtained after treatment exhibits a good bacteriostatic effect on foodborne pathogenic bacteria, and can be used as a drug against foodborne pathogenic bacteria in the pharmaceutical and food industries.

The foregoing description of the embodiments of the present disclosure has been presented for the purposes of illustration and description. Each and every page of this submission, and all contents thereon, however characterized, identified, or numbered, is considered a substantive part of this application for all purposes, irrespective of form or placement within the application. This specification is not intended to be exhaustive or to limit the present disclosure to the precise form disclosed. Many modifications and variations are possible in light of the present disclosure.

Although the present application is shown in a limited number of forms, the scope of the disclosure is not limited to just these forms, but is amenable to various changes and modifications. The present application does not explicitly recite all possible combinations of features that fall within the scope of the disclosure. The features disclosed herein for the various embodiments can generally be interchanged and combined into any combinations that are not self-contradictory without departing from the scope of the disclosure. In particular, the limitations presented in dependent claims below can be combined with their corresponding independent claims in any number and in any order without departing from the scope of this disclosure, unless the dependent claims are logically incompatible with each other.

What is claimed is:

1. A method of preparing a low-polarity Chenopodium quinoa saponin monomer, the method comprising:
S1
weighing Chenopodium quinoa bran;
subjecting the Chenopodium quinoa bran to heating treatment with superheated steam at a temperature of 110° C. to 120° C. for 1 hour to 2 hours;
air-drying a resulting heat-treated bran to have a water content of not greater than 10%;
crushing a resulting air-dried Chenopodium quinoa bran and sieving a resulting crushed Chenopodium quinoa bran by passing through a 40-mesh sieve;
subjecting a resulting sieved Chenopodium quinoa bran to extraction with 70% ethanol at 60° C. for 0.5 h;
subjecting an obtained extract to a first concentration in a vacuum decompression concentration tank until a concentrate has no alcohol taste;
subjecting the concentrate obtained from the first concentration to saponin purification by using HP-20 macroporous resin with a volume 15 times to 30 times that of the concentrate to obtain an eluate;
subjecting the eluate to a second concentration again in the vacuum decompression concentration tank and recovering ethanol, then spray-drying a concentrated solution obtained from the second concentration at a temperature of 100° C. to 120° C. to obtain a Chenopodium quinoa saponin mixture; and
S2
subjecting the Chenopodium quinoa saponin mixture obtained in S1 to gradient elution with a 20% aqueous methanol solution, a 40% aqueous methanol solution, and an 80% aqueous methanol solution sequentially through a reverse-phase chromatographic column;
collecting an eluate after elution with the 80% aqueous methanol solution;
and
concentrating and drying the eluate by using a rotary evaporator and recovering methanol, obtaining the low-polarity Chenopodium quinoa saponin monomer, namely a phytolaccagenic acid with a purity of not less than 90%.

2. The method of preparing the low-polarity Chenopodium quinoa saponin monomer of claim 1, wherein in S1, an amount of the 70% ethanol is 10 times to 15 times a mass of the resulting crushed Chenopodium quinoa bran.

3. The method of preparing the low-polarity Chenopodium quinoa saponin monomer of claim 1, wherein:
in S1, during the first concentration, the vacuum decompression concentration tank is at a pressure of 0.09 MPa and a temperature of 40° C. to 50° C.; and
during the second concentration, the vacuum decompression concentration tank is at a pressure of 0.09 MPa and a temperature of 40° C. to 50° C.

4. The method of preparing the low-polarity Chenopodium quinoa saponin monomer of claim 1, wherein in S1, the saponin purification is conducted by:
injecting the concentrate obtained from the first concentration into a chromatographic column filled with the HP-20 macroporous resin;
standing and adsorbing for 8 hours to 12 hours;
rinsing the chromatographic column with 2 BV of distilled water;
rinsing the chromatographic column with 2 BV of 95% aqueous ethanol solution; and
collecting the eluate.

5. The method of preparing the low-polarity Chenopodium quinoa saponin monomer of claim 1, wherein in S2, the reverse-phase chromatographic column is filled with a reverse-phase silica gel packing, a filling amount of the reverse-phase silica gel packing being 15 times to 30 times a mass of the Chenopodium quinoa saponin mixture.

6. The method of preparing the low-polarity Chenopodium quinoa saponin monomer of claim 1, wherein in S2, the rotary evaporator is at a temperature of 40° C. to 60° C. and a pressure of 0.09 MPa.
